(19) United States
(12) Patent Application Publication
(10) Pub. No.: US 2024/0082108 A1
(43) Pub. Date: Mar. 14, 2024

(54) SMART PILL DISPENSER

(71) Applicant: THE JOHNS HOPKINS UNIVERSITY, Baltimore, MD (US)

(72) Inventors: Andrea Gielen, Ellicott City, MD (US); Soraya J. Bailey, Baltimore, MD (US); Eileen M. McDonald, Baltimore, MD (US); Nathan W. Scott, Baltimore, MD (US); Christopher Dykes, Baltimore, MD (US)

(21) Appl. No.: 18/263,644

(22) PCT Filed: Jan. 31, 2022
(86) PCT No.: PCT/US2022/014584

§ 371 Date: Jul. 31, 2023

Related U.S. Application Data
(60) Provisional application No. 63/143,602, filed on Jan. 29, 2021.

(51) Int. Cl. A61J 1/14; A61J 1/03; A61J 7/00; A61J 7/04

(52) U.S. Cl. CPC A61J 1/1437; A61J 1/03; A61J 7/0076; A61J 7/0481; A61J 2200/70

(57) ABSTRACT

Prescription medications, especially opioids, are often abused. The present invention provides a pill dispensing device that is designed to reduce misuse, diversion, and pilfering or prescribed opioids or other medications that may require regulated dispensing. The device of the present invention securely holds a repository of pills and dispenses a single pill only after the allotted prescribed time interval and only after verification of the correct user through the biometric ID device. The dispensing device includes a locked tamper-resistant outer housing, a biometric ID device, a display for alerting the user to critical information, (such as time to the next pill and number of pills remaining) and a secure mechanical pill dispensing mechanism.

### CROSS REFERENCE TO RELATED APPLICATIONS

This application claims the benefit of U.S. Provisional Patent Application No. 63/143,602 filed on Jan. 29, 2021, which is incorporated by reference, herein, in its entirety.

### GOVERNMENT SUPPORT

This invention was made with government support under CE002466 awarded by the National Institutes of Health. The government has certain rights in the invention.

### FIELD OF THE INVENTION

The present invention relates generally to medical devices. More particularly the present invention relates to a personalized, tamper-resistant pill dispenser.

### BACKGROUND OF THE INVENTION

In the U.S., 67,367 drug overdose deaths occurred in 2018. Opioids were involved in nearly 70% of overdose deaths, and of those opioid related deaths, two out of three involved synthetic opioids (excluding methadone). 80% of heroin addicts report first using a friend or family member's prescription pain medication. 6 in 10 adults who reported getting a prescription opioid in the past year reported having ‘left over’ medication. Only 29% of these homes safely stored their medications (33% with 0-7 yo, 12% with 8-18 yo).

These facts show that this is a huge problem, and there is a need for a device that helps reduce the amount of overdose deaths from prescription opioids. Products currently available are costly and cumbersome. In addition, products currently available are not completely tamper proof, with materials and locks that can be broken and breached. Attackers/Un-authorized users are often teenagers/young adults using household tools to attempt to break into device.

Therefore, it would be advantageous to provide a personalized, tamper-resistant pill dispenser, and methods of using the same.

### SUMMARY OF THE INVENTION

The foregoing needs are met by the present invention, which provides a device including a tamper-resistant outer housing. The device includes a display and a biometric scanner. The device includes an interior assembly including a pill bottle, a chute for transmitting a pill to a user, and a mechanism for dispensing a single pill from a plurality of pills. The mechanism for dispensing a pill defines an opening configured to engage the pill in the pill bottle. The mechanism for dispensing a single pill is actuated to transfer the pill to the chute. The device also includes a locking mechanism to secure the interior assembly within the tamper-resistant outer housing.

In accordance with an aspect of the present invention, the biometric scanner takes the form of a fingerprint scanner. The tamper resistant outer housing is formed from one selected from a group consisting of nylon, metal, and polycarbonate. The device includes a programming means for programming the device. The programming means includes a plug for connecting the device to a programming device. The plug is concealed behind the outer housing when the device is fully assembled. The programming device can be one selected from a group of a smartphone, tablet, laptop, dedicated computing device, and personal computer. The device includes an application for programming the device to dispense the pill at a predetermined time interval. The application prompts a pharmacist to enter relevant information about the user and a prescription of the user. The display is configured to show the user a number of pills remaining and an amount of time until a next pill is dispensed. The locking mechanism includes a tamper-resistant lock and key.

In accordance with another aspect of the present invention, a device includes a tamper-resistant outer housing, a display, and a biometric scanner. The device also includes an interior assembly having a pill bottle and a pill wheel defining a hole sized for a single pill. The interior assembly also includes a chute for transmitting the single pill to a user. The interior assembly also includes a mechanism for dispensing the single pill from a plurality of pills. The mechanism for dispensing the single pill is actuated to transfer the single pill to the chute. Additionally, the device includes a locking mechanism to secure the interior assembly within the tamper-resistant outer housing.

### BRIEF DESCRIPTION OF THE FIGURES

FIGS. 1A-1D illustrate perspective and semi-sectional views of an outer housing of a pill dispensing device, according to an embodiment of the present invention.

FIG. 2 illustrates an exploded view of the internal assembly and the protective outer metal housing of a pill dispenser, according to an embodiment of the present invention.

FIGS. 3A-3C illustrate exploded and perspective views of an electronics head and frame assembly of a pill dispensing device, according to an embodiment of the present invention.

FIGS. 4A and 4B illustrate partially sectional and sectional views of a pill dispenser, according to an embodiment of the present invention.

FIGS. 5A-5C illustrate partially sectional, exploded, and zoomed-in views of an internal assembly of a pill dispenser according to an embodiment of the present invention.

FIG. 6A illustrates a perspective view of the internal pill dispenser, according to an embodiment of the present invention.

FIG. 6B illustrates an exploded view of the internal pill dispenser and frame, according to an embodiment of the present invention.

FIGS. 7A-7C illustrate zoomed-in and exploded views of an initial removal of the pill bottle, according to an embodiment of the present invention.

FIGS. 8A and 8B illustrate perspective and zoomed-in views of an initial replacement of the pill bottle, according to an embodiment of the present invention.

FIGS. 9A and 9B illustrate perspective and exploded views of loading the medication in the pill bottle, according to an embodiment of the present invention.

FIGS. 10A-10D illustrate perspective views of an initial power up of the pill dispenser of the present invention.

FIGS. 11A-11D illustrates perspective views of programming a pill dispensing device, according to an embodiment of the present invention.

FIG. 12 illustrates a perspective view of a pill dispensing device ready for fingerprint programming by the user, according to the embodiment of the present invention.

FIG. 13 illustrate s perspective view of a pill dispensing device during the pill dispensing process, according to an embodiment of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Prescription medications, especially opioids, are often abused. The present invention provides a pill dispensing device that is designed to reduce misuse, diversion, and pilfering or prescribed opioids or other medications that may require regulated dispensing. The device of the present invention includes a tamper-resistant outer housing, a biometric scanner, and a display for alerting the user to critical information, such as time to the next pill, number of pills remaining, and battery life of the device. The device also includes an internal mechanism designed to store the medication and dispense one pill at each determined interval.

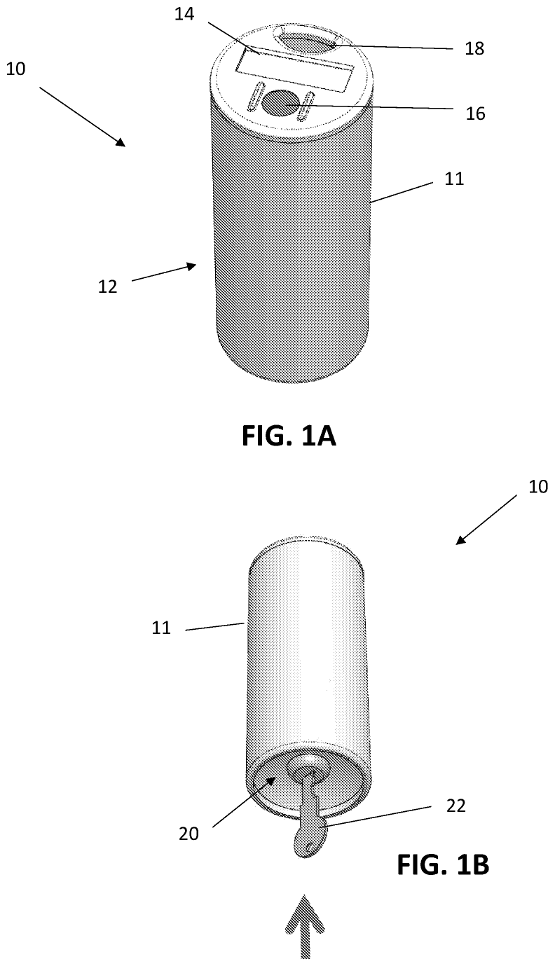

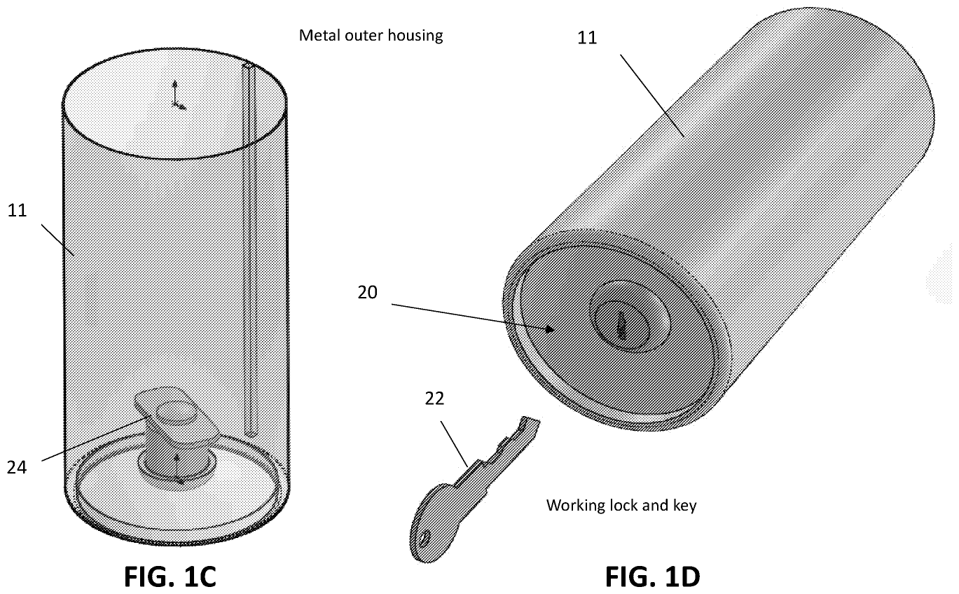

FIGS. 1A-1D illustrate perspective and semi-sectional views of an outer housing of a pill dispensing device, according to an embodiment of the present invention. The outer housing 12 of the pill dispensing device 10 is designed to be tamper-resistant and tamper-evident. The device 10 includes an outer sheath 11, a display 14, a fingerprint scanner 16, and a slot 18 for dispensing a pill. While a fingerprint scanner is described herein as an illustration of the present invention, it is to be understood that any other biometric security device known to or conceivable to one of skill in the art could also be used.

As illustrated in FIGS. 1B-1D, the pill dispensing device 10 includes a locking mechanism 20. The actuation of the locking mechanism 20 allows the device 10 to be opened and closed for filling and refilling by a pharmacist or authorized user. As illustrated in FIGS. 1B-1D, the locking mechanism takes the form of a lock and key 22. The locking mechanism 20 can take the form of any locking mechanism known to or conceivable to one of skill in the art. The locking mechanism 20 can also take the form of a tamper resistant and/or tamper evident lock. Additionally, the locking mechanism 20 can take the form of a mechanism that is difficult to pick or duplicate a key for the lock. The key 22 for the locking mechanism 20 can take the form of a key format that is only available to pharmacists for opening the device. 10. The internal component 24 of the locking mechanism 20 holds the protective outer housing 12 in place. The outer housing 12, and in particular the outer sheath 11 of the outer housing 12, can be formed from a strong and tamper-resistant material such as metal, Lexan, or a polycarbonate. Alternately, the outer housing 12 can be formed from any other strong tamper resistant material known to or conceivable to one of skill in the art.

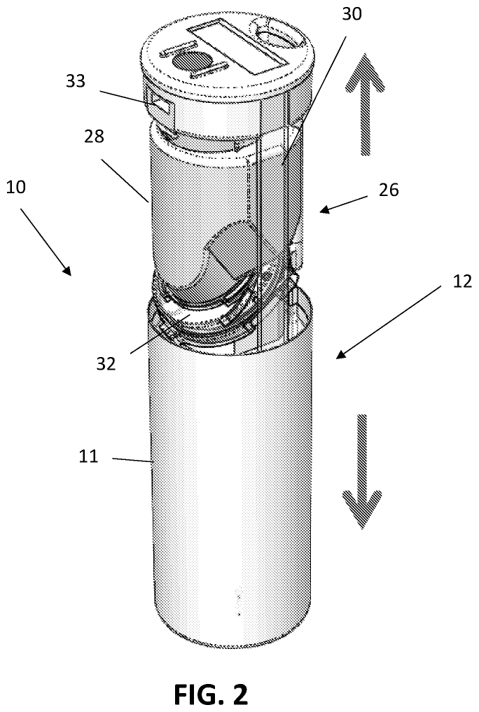

FIG. 2 illustrates an exploded view of the internal assembly and the protective outer metal housing of a pill dispenser, according to an embodiment of the present invention. As illustrated in FIG. 2, the device 10 includes the protective outer housing 12 and an internal assembly 26. The internal assembly 26 is coupled to the outer housing 12 using the locking mechanism, as will be described further herein. The inner assembly includes a pill bottle 28, a support frame 30, and a pill dispensing mechanism 32. The outer housing 12 can also include charging and information port 33.

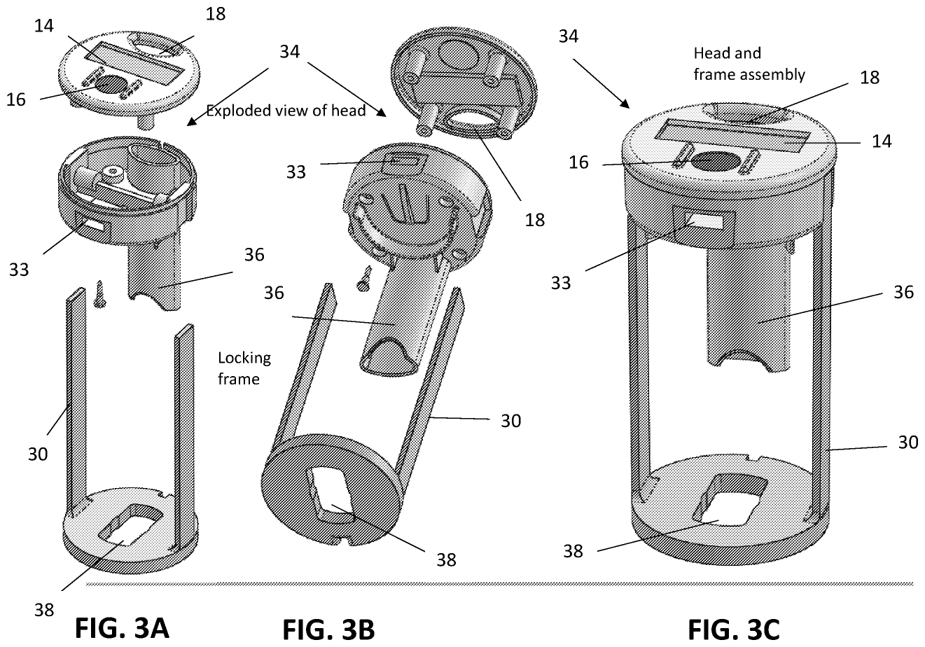

FIGS. 3A-3C illustrate exploded and perspective views of an electronics head and frame assembly of a pill dispensing device, according to an embodiment of the present invention. The electronics head 34 houses the display 14, the fingerprint scanner 16, and their associated electronics, including, but not limited to, wiring, microprocessors, communication, power, and other componentry. The electronics head 34 can include a chute 36 for dispensing the pill out of the slot 18, when it is authorized. The electronics head 34 couples to the support frame 30. The support frame 30 defines an opening 38 to engage with the locking mechanism of the outer housing. The electronics head 34 and support frame 30 can be formed from metal or any other strong and tamper resistant or tramper evident material known to or conceivable to one of skill in the art. In some embodiments the charging and information port 33, can be disposed on the electronics head 34 such that it is concealed by the outer housing, when the entire device is assembled. The power source can take the form of a battery, rechargeable battery, AC or DC power source, or any other power source known to or conceivable to one of skill in the art.

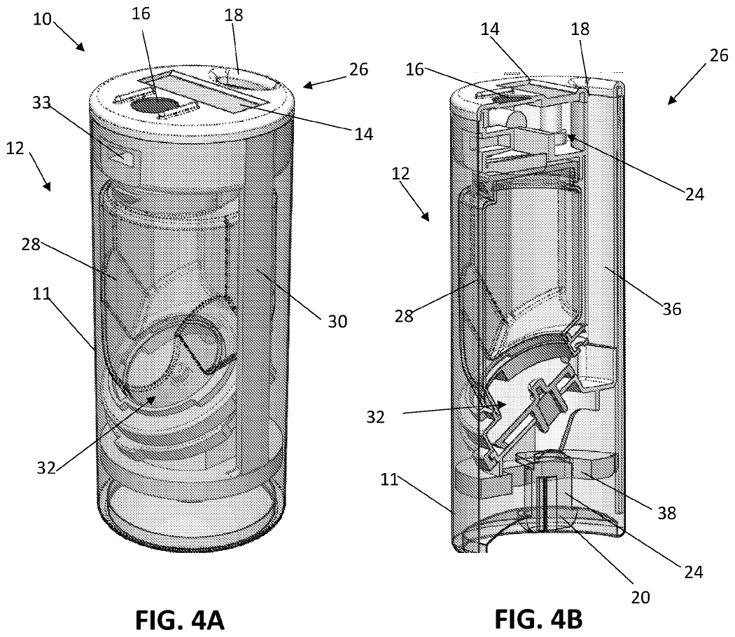

FIGS. 4A and 4B illustrate partially sectional and sectional views of a pill dispenser, according to an embodiment of the present invention. The outer housing 12 includes the outer sheath 11, display 14, the fingerprint scanner 16, and the slot 18 for dispensing a pill. The outer housing 12 can also include the charging port 33 for rechargeable implementations of the device. The pill dispensing device 10 includes a locking mechanism 20. The actuation of the locking mechanism 20 allows the device 10 to be opened and closed for filling and refilling by a pharmacist or authorized user. The internal component 24 of the locking mechanism 20 holds the protective outer housing 12 in place, by coupling the outer housing 12 to the support frame 30. This mechanism secures the contents of the device against unauthorized use. The pill bottle 28 and the pill dispensing mechanism 32 are coupled to the support frame 30.

Further, as illustrated in FIGS. 4A and 4B, the electronics head 34 houses the display 14, the fingerprint scanner 16, and their associated electronics, including, but not limited to, wiring, microprocessors, communication, power, and other componentry. The electronics head 34 also couples to the support frame 30. The support frame 30 defines an opening 38 to engage with the locking mechanism of the outer housing. The electronics head 34 and support frame 30 can be formed from metal or any other strong and tamper resistant or tramper evident material known to or conceivable to one of skill in the art. The electronics head 34 can include the chute 36 for dispensing the pill out of the slot 18, when it is authorized. A pill is dispensed from the pill bottle 28 via rotation of the pill dispensing mechanism 32, in order to align a pill with the chute 36. The electronics and motor for actuating the pill dispensing mechanism 32 can be disposed within the electronics head 34. Alternately, the motor can be positioned below the pill dispensing mechanism 32. Once the pill is dispensed into the chute, the user can shake the pill into a hand via slot 18.

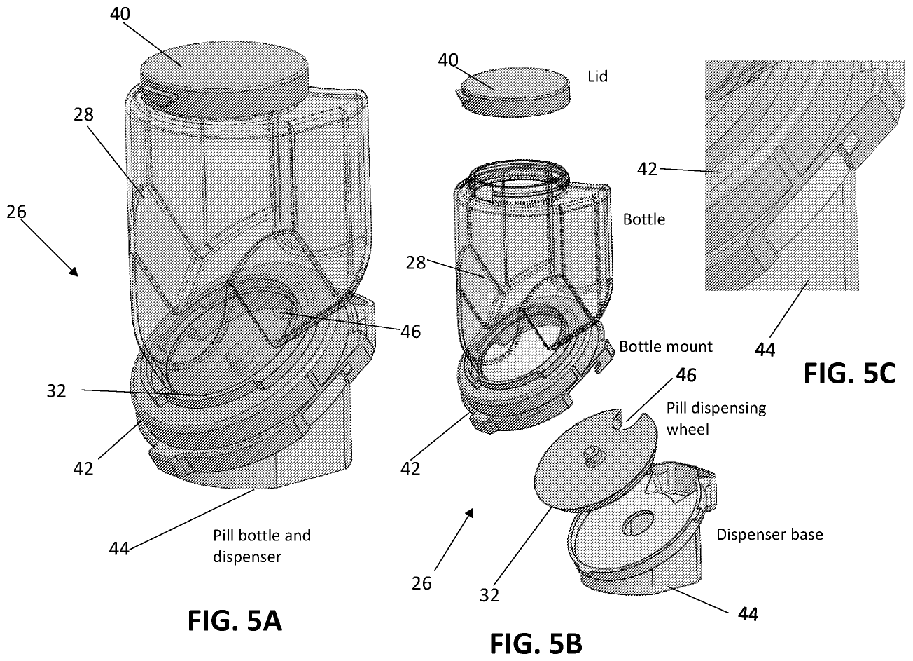

FIGS. 5A-5C illustrate partially sectional, exploded, and zoomed-in views of an internal assembly of a pill dispenser according to an embodiment of the present invention. The internal assembly 26 includes a pill bottle 28, a support frame 30, and a pill dispensing mechanism 32. Further, the internal assembly 26 includes a lid 40, a bottle mount 42, and dispenser base 44. The lid 40 couples to the pill bottle 28. The coupling between the lid 40 and bottle 28 can be pressure fit, thread and groove, or any other suitable means of coupling known to or conceivable to one of skill in the art. The bottle mount 42 is disposed at the bottom of the bottle 28. The bottle mount 42 couples to the dispenser base 44. The coupling between the bottle mount 42 and the dispenser base 44, is illustrated as a tab and groove coupling. However, any suitable coupling known to or conceivable to one of skill in the art could also be used. Alternately, the bottle mount and the dispenser base can be permanently coupled together. The pill dispensing mechanism 32 is sandwiched between the bottle mount 42 and the dispenser base 44. As illustrated in FIGS. 5A and 5B, the pill dispensing mechanism takes the form of a pill dispensing wheel defining an opening 46. The opening 46 engages a pill in the pill bottle 28. The wheel is then actuated to sweep the pill into the chute for dispensing to the user. In embodiments where a pill wheel is used, the pill wheel can be chosen for each user, based on the size and shape of their particular pill to be dispensed. Any pill dispensing mechanism known to or conceivable to one of skill in the art, such as a cartridge can also be used. In some embodiments a motor for actuating the pill dispensing mechanism 32 is disposed below the dispenser base 44.

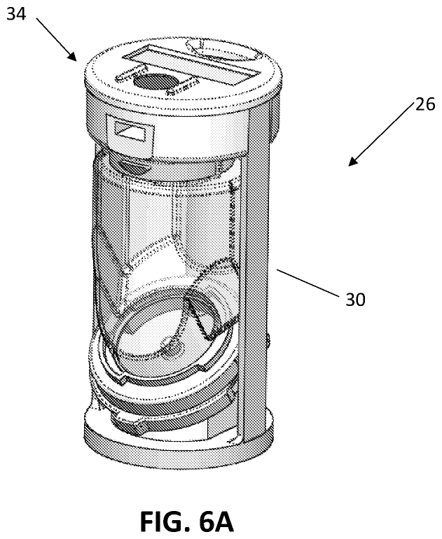

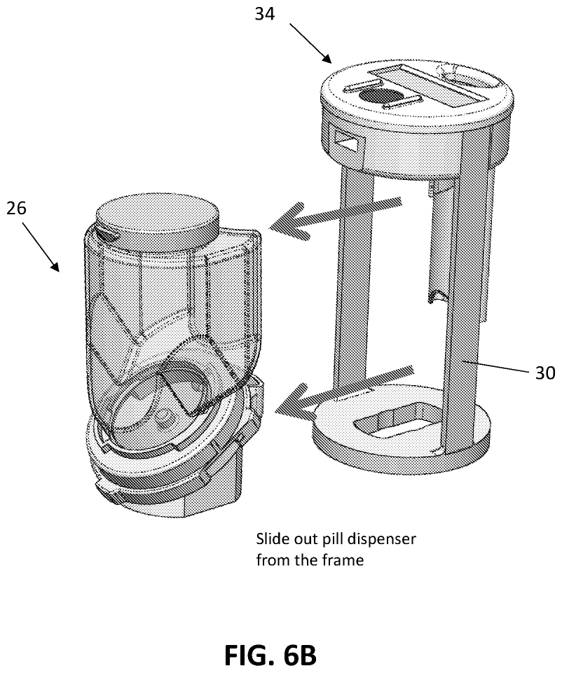

FIG. 6A illustrates a perspective view of the internal pill dispenser, according to an embodiment of the present invention. FIG. 6B illustrates an exploded view of the internal pill dispenser and frame, according to an embodiment of the present invention. FIGS. 6A and 6B illustrate the internal assembly 26 coupled to the support frame 30 and the electronics head 34. The electronics head 34, support frame 30, and internal assembly 26 are disposable within the outer housing, as described further herein. The internal assembly 26 can slide in and out of the support frame 30, as illustrated in FIG. 6B.

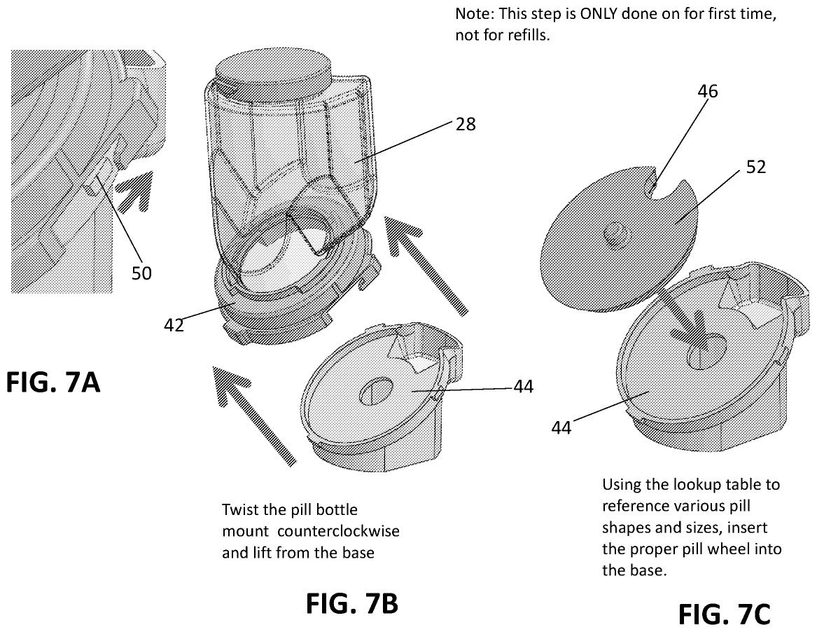

FIGS. 7A-7C illustrate zoomed-in and exploded views of an initial removal of the pill bottle as well as an initialization workflow for the pill dispenser, according to an embodiment of the present invention. As illustrated in FIGS. 7A-7C, the first time the pill dispenser is used for a particular user and prescription, initialization steps are taken to prepare the device for the particular user. The pill bottle 28 is disengaged from the dispenser base 44 via bottle mount 42. Here, the pill bottle 28 is twisted counter-clockwise to disengage the tab and grove coupling 50. Using a pharmacist lookup table associated with the device to reference various pill shapes and sizes, the pharmacist can insert the proper pill wheel 50 as the pill dispensing mechanism into the base. The proper pill wheel 52 will define an opening 46 that is the appropriate size for the particular medication. This step only needs to be completed for the first dispensing of the prescription and not for refills. If another pill dispensing mechanism is used, this process may vary slightly based on the pill dispensing mechanism used. Any pill dispensing mechanism known to or conceivable to one of skill in the art, as well as the associated initialization process are understood to be included herein.

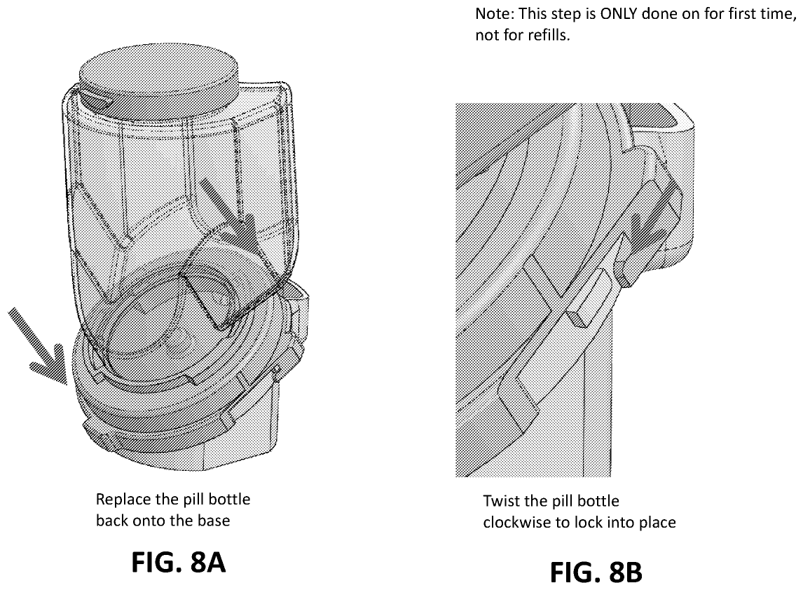

FIGS. 8A and 8B illustrate perspective and zoomed-in views of an initial replacement of the pill bottle, as well as an initialization workflow for the pill dispenser, according to an embodiment of the present invention. As illustrated in FIGS. 8A and 8B the bottle is replaced on the dispenser base and the coupling between the dispenser base and the bottle mount is re-engaged. The pill bottle is twisted clockwise to re-engage the coupling. As noted above, this process is only necessary for the first time the pill dispenser is used for a particular user and prescription.

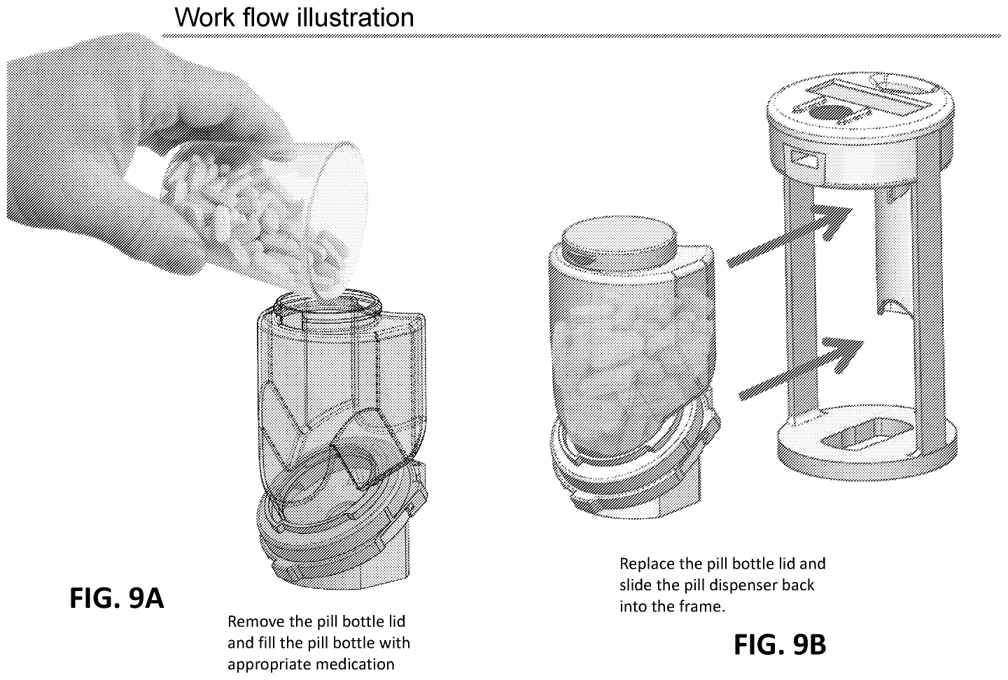

FIGS. 9A and 9B illustrate perspective and exploded views of loading the medication in the pill bottle and the associated workflow, according to an embodiment of the present invention. As illustrated in FIGS. 9A and 9B, when the pill bottle is removed, the pill bottle can be filled with the user's prescription medication. The lid is removed to place the medication in the pill bottle and replaced when the medication is loaded. The internal assembly is returned to the frame such that it can be placed and locked within the outer housing.

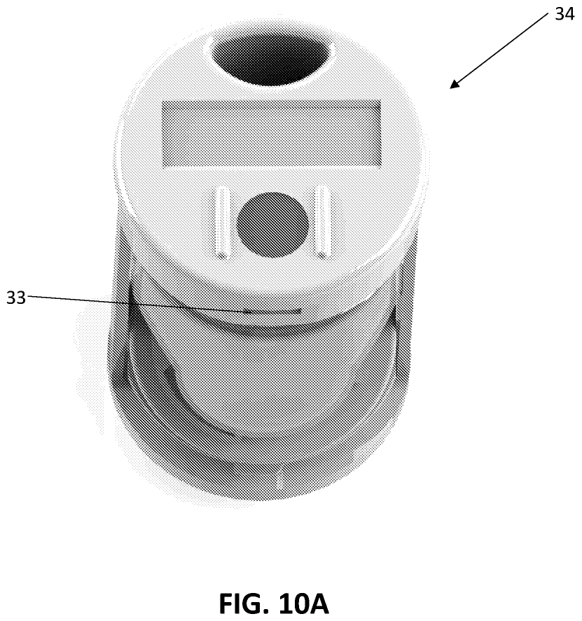

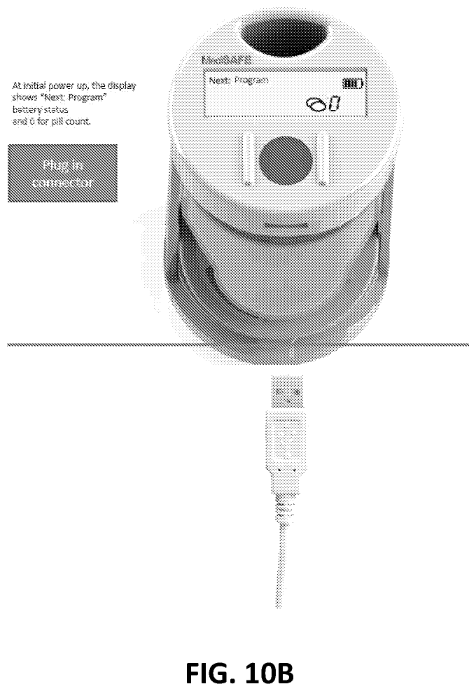

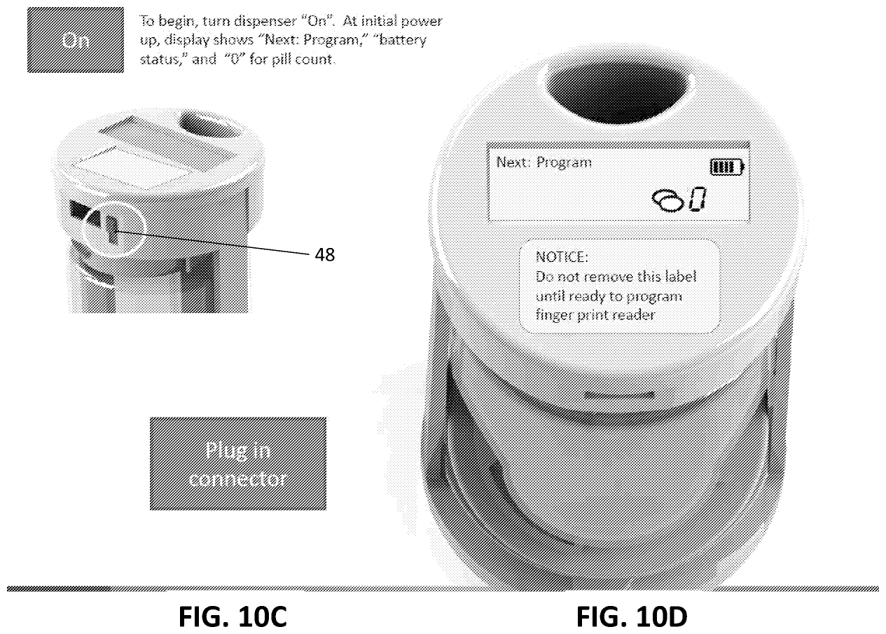

FIGS. 10A-10D illustrate perspective views of an initial power up of the pill dispenser of the present invention. The electronics head 34 includes a charging and communications port 33. The port can be positioned such that it is concealed by the outer housing when the device is fully assembled. In other embodiments, the port may take the form of a separate communications port that is concealed and a charging port that is exposed. At initial power up by the pharmacist, the pharmacist plugs the charging and communications port into a source of power and/or a device for programming. After the device is plugged in the display shows instructional and information text such as, but not limited to: “next: program”, battery status, and 0 for pill count. In some instances, there can be a physical power on switch 48, as illustrated in FIG. 10C. In other instances, the device will automatically turn on when it is connected to a source of power.

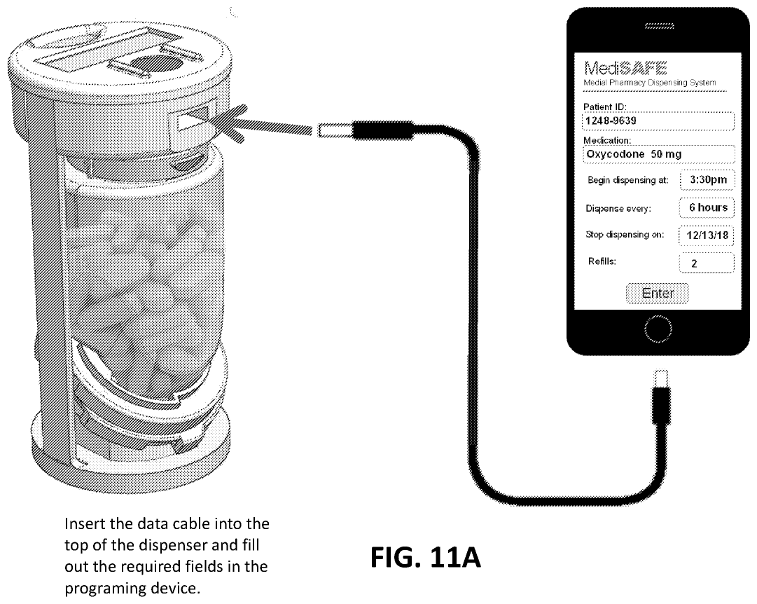

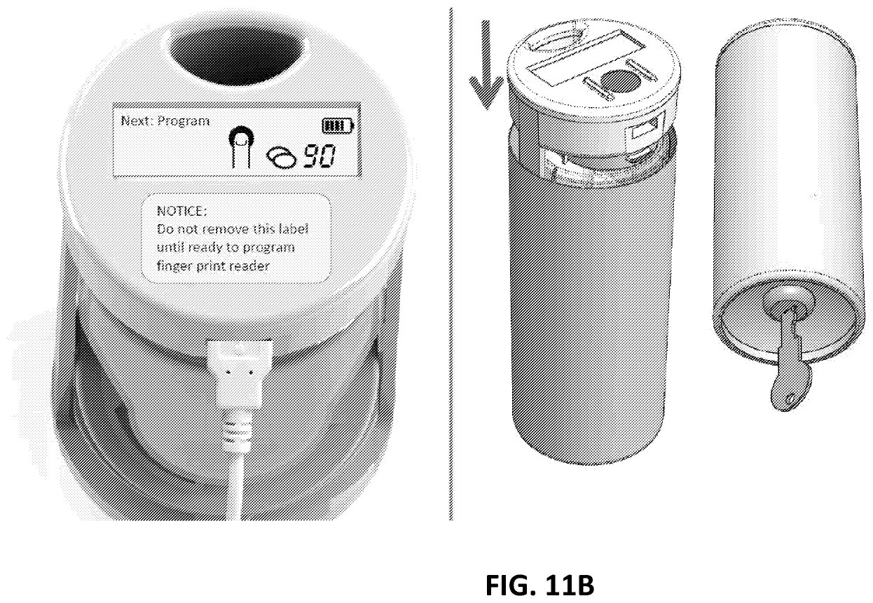

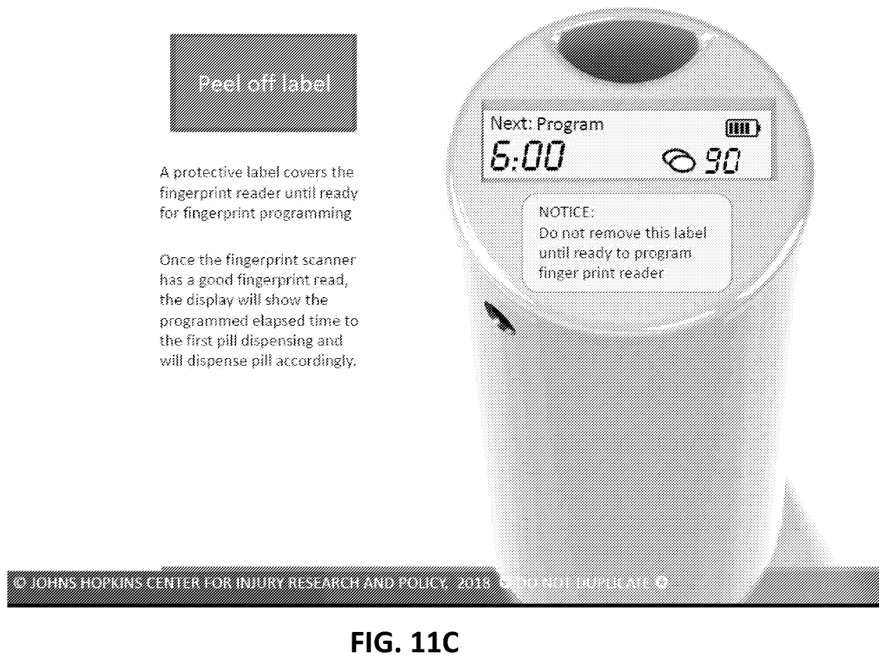

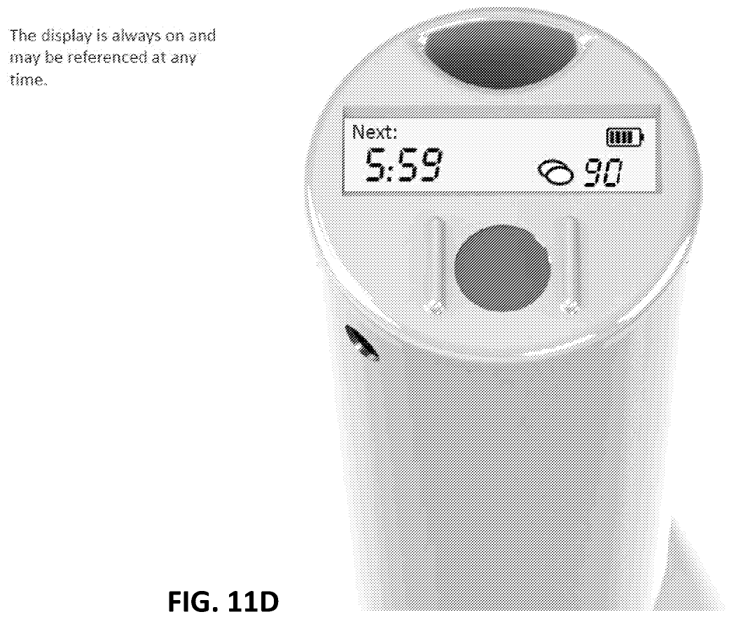

FIGS. 11A-11D illustrates perspective views of programming a pill dispensing device, according to an embodiment of the present invention. In some embodiments the device is programmed via a programming device, such as a smart phone, tablet, PC, laptop, dedicated computing device, or other programming device known to or conceivable to one of skill in the art. In other embodiments, a microprocessor capable of allowing the pharmacist to program the device can be disposed in the electronics head and the pharmacist can program the device via the display and/or buttons positioned on the outside of the electronics head. Alternately, the device can be programed via the display and/or buttons positioned such that the programming controls are locked from access during normal use by the prescription holder. FIG. 11A illustrates an embodiment where the device is programmed via a smartphone. An application on the smartphone allows the pharmacist to enter the necessary data, prescription information, instructions for the user, and any other relevant information known to or conceivable to one of skill in the art. FIG. 11B illustrates the programmed device and the placement of and locking of the internal assembly into the outer housing. FIG. 11C illustrates a device that is ready for biometric programming. As illustrated in FIG. 11C, the device calls for a fingerprint to dispense the medication. In some embodiments, a protective label covers the fingerprint reader until the device is ready for fingerprint programming. Once the fingerprint scanner has a good fingerprint read from the intended user, the display will show the programmed elapsed time to the first pill dispensing and will dispense pills according, after the authorized fingerprint is applied to the scanner. Once the fingerprint scanner is programmed, FIG. 11D illustrates that the device display is always on. The device display shows information relevant to the user, such as time to next pill, battery life, and number of remaining pills.

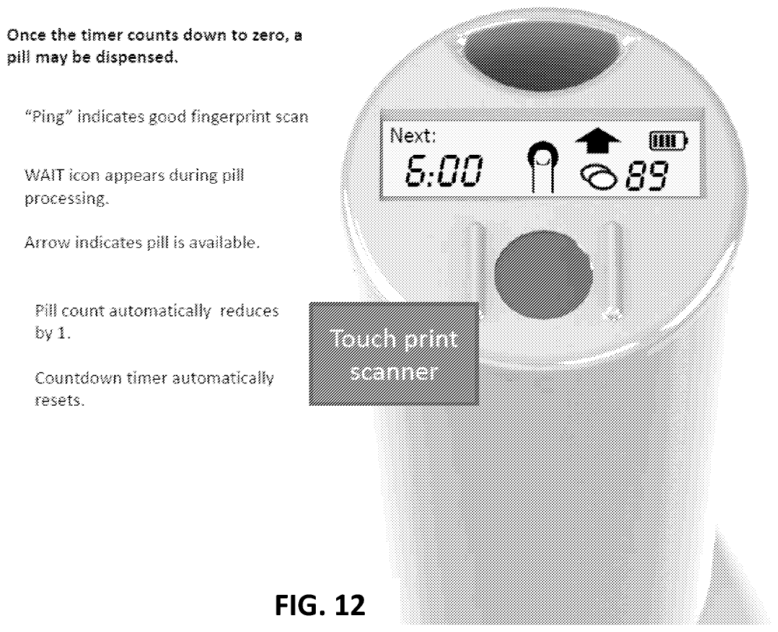

FIG. 12 illustrates a perspective view of a pill dispensing device ready for fingerprint programming by the user, according to the embodiment of the present invention. Once the timer counts down to zero, a pill can be dispensed to the authorized user. The display can indicate that the authorized user should touch the fingerprint scanner. An audio, haptic, light based, or other feedback to the user indicates that there was a good fingerprint scan or can let the user know to try again. An icon or language that indicates that the user wait can be displayed during processing. During processing, the motor of the device actuates the pill wheel to engage a pill and align the pill with the chute, such that it can be dispensed from the slot. The display can then indicate to the user that the pill is available. In FIG. 12, an arrow is used to indicate that the pill is available. The arrow as indicator is not meant to be considered limiting, and the user can be alerted to the pill that is ready in any number of ways known to or conceivable to one of skill in the art, such as sound, haptic, light, or other alert on the display. The pill count displayed is automatically reduced by one. The countdown timer automatically resets. The display, countdown, and other visual indications are made possible by the microprocessor disposed in the electronics head.

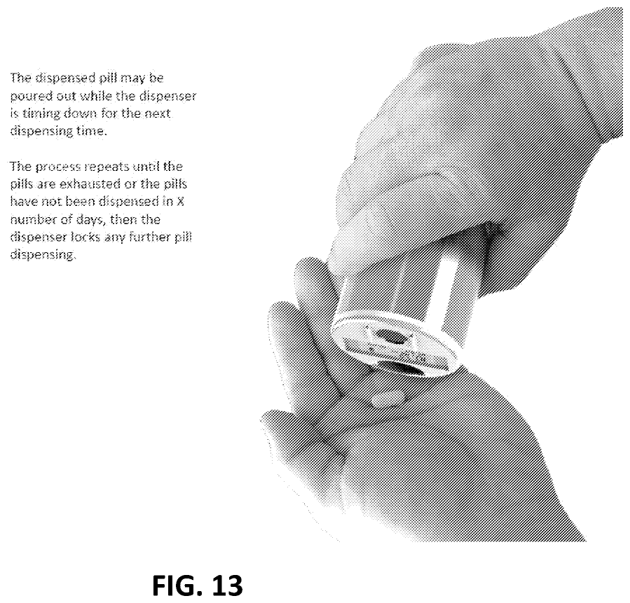

FIG. 13 illustrate a perspective view of a pill dispensing device during the pill dispensing process, according to an embodiment of the present invention. As illustrated in FIG. 13, the dispensed pill is poured out of the dispenser, while the dispenser begins counting down to the next time authorized for dispensing. The process repeats until the pills are exhausted. In addition, if the pills have not been dispensed in X number of days or the dispenser is tampered with, or an unauthorized user repeatedly attempts to dispense the medication, the dispenser locks any further pill dispensing. The dispenser can also include a sensor in the lock to indicate attempts to destroy or pick the lock that would then trigger the dispenser to be locked.

It should be noted that the communications protocols described herein can be executed with a program(s) fixed on one or more non-transitory computer readable medium. The non-transitory computer readable medium can be loaded onto a computing device, server, imaging device processor, smartphone, tablet, phablet, or any other suitable device known to or conceivable by one of skill in the art.

It should also be noted that herein any computing necessary can be carried out using a computer, non-transitory computer readable medium, or alternately a computing device, microprocessor, or other computer type device independent of or incorporated with the present invention. An independent computing device can be networked together with the device either with wires or wirelessly. Indeed, any suitable method of programming and actuation of the device known to or conceivable by one of skill in the art could be used.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A device comprising:
a tamper-resistant outer housing;
a display;
a biometric scanner;
an interior assembly comprising:
a pill bottle;
a chute for transmitting a single pill to a user;
a mechanism for dispensing the single pill from a plurality of pills defining an opening configured to engage the single pill in the pill bottle, wherein the mechanism for dispensing the single pill is actuated to transfer the single pill to the chute; and,
a locking mechanism to secure the interior assembly within the tamper-resistant outer housing.

2. The device of claim 1 wherein the biometric scanner comprises a fingerprint scanner.

3. The device of claim 1 wherein the tamper-resistant outer housing is formed from one selected from a group consisting of nylon, metal, and polycarbonate.

4. The device of claim 1 further comprising a programming means for programming the device.

5. The device of claim 4 wherein the programming means comprises a plug for connecting the device to a programming device.

6. The device of claim 5 wherein the plug is concealed behind the outer housing when the device is fully assembled.

7. The device of claim 5, wherein the programming device comprises one selected from a group consisting of a smartphone, tablet, laptop, dedicated computing device, and personal computer.

8. The device of claim 7 further comprising an application for programming the device to dispense the pill at a predetermined time interval.

9. The device of claim 8 wherein the application prompts a pharmacist to enter relevant information about the user and a prescription of the user.

10. The device of claim 1 wherein the display is configured to show the user a number of pills remaining and an amount of time until a next pill is dispensed.

11. The device of claim 1 wherein the locking mechanism comprises a tamper-resistant lock and key.

12. A device comprising:
a tamper-resistant outer housing;
a display;
a biometric scanner; and
an interior assembly comprising:
a pill bottle;
a pill wheel defining a hole sized for a single pill;
a chute for transmitting the single pill to a user;
a mechanism for dispensing the single pill from a plurality of pills, wherein the mechanism for dispensing the single pill is actuated to transfer the single pill to the chute; and
a locking mechanism to secure the interior assembly within the tamper-resistant outer housing.

13. The device of claim 12 wherein the biometric scanner comprises a fingerprint scanner.

14. The device of claim 12 wherein the tamper-resistant outer housing is formed from one selected from a group consisting of nylon, metal, and polycarbonate.

15. The device of claim 12 further comprising a programming means for programming the device.

16. The device of claim 15 wherein the programming means comprises a plug for connecting the device to a programming device.

17. The device of claim 16 wherein the plug is concealed behind the outer housing when the device is fully assembled.

18. The device of claim 16, wherein the programming device comprises one selected from a group consisting of a smartphone, tablet, laptop, dedicated computing device, and personal computer.

19. The device of claim 18 further comprising an application for programming the device to dispense the pill at a predetermined time interval.

20. The device of claim 19 wherein the application prompts a pharmacist to enter relevant information about the user and a prescription of the user.

21. The device of claim 12 wherein the display is configured to show the user a number of pills remaining and an amount of time until a next pill is dispensed.

22. The device of claim 12 wherein the locking mechanism comprises a tamper-resistant lock and key.
